(19) United States
(12) Patent Application Publication
(10) Pub. No.: US 2023/0189904 A1
(43) Pub. Date: Jun. 22, 2023

(54) APPARATUS AND METHOD FOR PROVIDING A SANITARY BARRIER FOR A USER OF AN OPTICAL ASSEMBLY

(71) Applicant: OCULUS SURGICAL, INC., Port St. Lucie, FL (US)

(72) Inventors: Michael ANNEN, Ft. Pierce, FL (US); William O'Brien, Port St. Lucie, FL (US)

(21) Appl. No.: 17/923,351

(22) PCT Filed: May 4, 2021
(86) PCT No.: PCT/US2021/030630

§ 371 Date: Nov. 4, 2022

Related U.S. Application Data
(60) Provisional application No. 63/019,593, filed on May 4, 2020.

(51) Int. Cl. A41D 13/11; A61B 3/00

(52) U.S. Cl. CPC A41D 13/1184; A61B 3/0083

(57) ABSTRACT

A method and apparatus is provided for providing a barrier for a user of an optical assembly. The optical assembly includes headgear and an optic within a housing. The apparatus includes the barrier including a protective shield defining an opening. The apparatus also includes a mounting fixture configured to attach the protective shield to the optical assembly such that the opening is aligned with an optical axis of the optic to provide an unobstructed path for light to the optic through the opening. The apparatus also includes an adaptive coupling positioned in a gap between the protective shield and the housing and configured to engage the protective shield adjacent the opening to form an airtight seal in the gap between the protective shield and the housing. A method is also provided for installing the apparatus to the optical assembly.

### CROSS-REFERENCE TO RELATED APPLICATIONS

This application claims priority to U.S. Provisional Application No. 63/019,593, filed May 4, 2020, under 35 U.S.C. § 120.

### BACKGROUND

Medical professionals use various optical devices (e.g. ophthalmoscope) to examine an eye of a patient. In particular, the ophthalmoscope is used by a medical professional to examine the retina of a patient. Based on the examination of the retina of the patient with the ophthalmoscope, the medical professional may diagnose one or more conditions and prescribe certain treatment, if appropriate.

### SUMMARY

Techniques are provided for a method and apparatus for providing a barrier (e.g. sanitary barrier) for a user of an optical assembly, e.g. an indirect ophthalmoscope. The inventors of the present invention recognized that although barriers are known, these barriers are deficient as they cannot provide an effective sanitary barrier for a user of an optical assembly. For example, U.S. Pat. No. 797,293 ('293 patent) discloses a permanent mounting of a plastic sheet on a separate headband placed around the head of the user and is used in conjunction with operating a vehicle. The inventors of the present invention recognized that the mounted sheet in the '293 has perforations to permit the free circulation of air though the sheet and thus does not provide an effective sanitary barrier to the wearer of the sheet against viral or bacterial pathogens that are in gaseous form (e.g. aerosol, droplets, etc.). Additionally, the inventors noted that conventional methods for mounting barriers to the head of a user of an optical assembly do not describe an effective method for allowing the line of sight of the user of the optical assembly to remain undisturbed, which is of significant importance when examining fine details (e.g. retina).

The inventors of the present invention recognized that conventional apparatuses are known, which feature a mounting fixture to mount a barrier to the optical assembly. However, the inventors recognized that these barriers do not feature an opening to permit light to pass unobstructed to the optic of the optical assembly, resulting in reduced visibility of the patient's eye. Additionally, the inventors realized that since there is no opening in these barriers and thus the user can view the patient through any portion of the barrier, the precise location where the mounting fixture secures the barrier to the optical assembly is not significant. Additionally, since there is no opening in these barriers, there is no motivation to provide a seal to prevent airborne pathogens from entering such an opening. The inventors developed an improved apparatus that features a barrier with an opening in front of the optic to permit unobstructed light to pass to the optical assembly. In order to seal the opening in the barrier and prevent transmission of airborne pathogens, the inventors designed a mounting fixture adjacent the opening that secures the barrier to the optical assembly while simultaneously providing such a seal. In some embodiments, the mounting fixture also features a self-centering feature which advantageously aligns the opening in the barrier with an optical axis of the optical assembly. This improves upon conventional apparatuses by providing an unobstructed view through the protective shield while simultaneously ensuring the sterile barrier is maintained between the user and the patient.

The inventors of the present invention recognized that although barriers are known for conventional optical assemblies, these barriers are deficient as they do not provide an effective sanitary barrier for the user and/or the patient. In another example, U.S. Pat. No. 5,341,513 ('513 patent) discloses a barrier for a user (e.g. surgeon) of a headlamp. The inventors of the present invention recognized that the disclosed barrier in the '513 patent is not a sanitary barrier that protects the user from viral or bacterial pathogens. Specifically, the barrier is designed to protect a surgeon from liquid splatter and thus does not teach an effective sanitary barrier to protect the user against viral or bacterial pathogens in a gaseous state (e.g. droplets, aerosol, etc.).

In a first set of embodiments, an apparatus is presented that provides a barrier for a user of an optical assembly including headgear and an optic within a housing. The apparatus includes the barrier including a protective shield that defines an opening. The apparatus also includes a mounting fixture configured to attach the protective shield to the optical assembly such that the opening is aligned with an optical axis of the optic to provide an unobstructed path for light to the optic through the opening. The apparatus also includes an adaptive coupling positioned in a gap between the protective shield and the housing and configured to engage the protective shield adjacent the opening to form an airtight seal in the gap between the protective shield and the housing.

In a second set of embodiments, a method is presented for installing the apparatus to the optical assembly. The method includes the step of attaching the mounting fixture to the optical assembly and subsequently attaching the protective shield to the mounting fixture such that the opening in the protective shield is aligned with the optical axis of the optical assembly.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is a schematic diagram that illustrates an example of an indirect ophthalmoscope being used to examine a patient;

FIG. 1B is an image that illustrates an example of an indirect ophthalmoscope being used to examine a patient;

FIG. 1C is an image that illustrates an example of an indirect ophthalmoscope;

FIG. 2A is an image that illustrates an example of an apparatus to provide a sanitary barrier for a user of an optical assembly, according to an embodiment;

FIG. 2B is an image that illustrates an example of a fastener strip of the apparatus of FIG. 2A, according to an embodiment;

FIG. 2C is an image that illustrates an example of components used to install the apparatus of FIG. 2A to the optical assembly, according to an embodiment;

FIG. 3A is an image that illustrates an example of the apparatus of FIG. 2A installed to an optical assembly and worn by a user in a first position, according to an embodiment;

FIG. 3B is an image that illustrates an example of the installed apparatus of FIG. 3A worn by a user in a second position, according to an embodiment;

FIG. 3C is an image that illustrates an example of the mounting bracket and adaptive coupling of the apparatus of FIG. 2A to engage the protective shield, according to an embodiment;

FIG. 3D is an image that illustrates an example of a top view of the installed apparatus of FIG. 3A, according to an embodiment;

FIG. 4A is an image that illustrates an example of a front perspective view of an apparatus to provide a sanitary barrier for a user of an optical assembly, according to an embodiment;

FIG. 4B is an image that illustrates an example of a side perspective view of a second portion of an adaptive coupling of the apparatus of FIG. 4A, according to an embodiment;

FIG. 4C is an image that illustrates an example of a top view of a second portion of an adaptive coupling secured to the sanitary barrier of the apparatus of FIG. 4A, according to an embodiment;

FIG. 4D is an image that illustrates an example of an exploded view of the apparatus of FIG. 4A, according to an embodiment;

FIG. 4E is an image that illustrates an example of a side view of the apparatus of FIG. 4A, according to an embodiment;

FIG. 4F is an image that illustrates an example of a front view of the apparatus of FIG. 4A, according to an embodiment;

FIG. 4G is an image that illustrates an example of a top view of the apparatus of FIG. 4A, according to an embodiment;

FIGS. 4H through 4K are images that illustrate examples of various exploded views of the apparatus of FIG. 4A, according to an embodiment;

FIG. 5A is a flow diagram that illustrates an example of a method for installing the apparatus of FIG. 2A to an optical assembly, according to an embodiment;

FIG. 5B is a flow diagram that illustrates an example of a method for installing the apparatus of FIG. 4A to an optical assembly, according to an embodiment; and

FIGS. 6A through 6S are images that illustrate an example of one or more steps of the method of the flow diagram of FIG. 5A, according to an embodiment.

### DETAILED DESCRIPTION

A method and apparatus are described for providing a barrier (e.g. sanitary barrier) for a user of an optical assembly. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of optical assemblies that include headgear, an optic within a housing and a fixture to mount the housing to the headgear. For purposes of this description, “optical assembly” means an optical device that is mounted to a head of a user, for purposes of one or more medical applications. In one embodiment, the optical assembly is a binocular indirect ophthalmoscope. In other embodiments, the optical assembly is a dental or surgical loupe, a surgical headlamp, Ultraviolet (UV) curing lamps and surgical lasers. In other embodiments, the optical assembly is utilized in military applications (e.g. night vision goggles, binoculars, etc.); jewelry/handcrafting applications (e.g. wearable loupes); or any application where the function of the optical assembly is impeded by the presence of a plastic or protective shield (e.g. due to reflection, attenuation, transparency, contrast, scattering, etc.) such as but not limited to diagnostic purposes (e.g. light in the visible and invisible spectrum), sound waves (e.g. ultrasound) or therapeutic purposes (e.g. a laser or any other type of electromagnetic radiation, etc.).

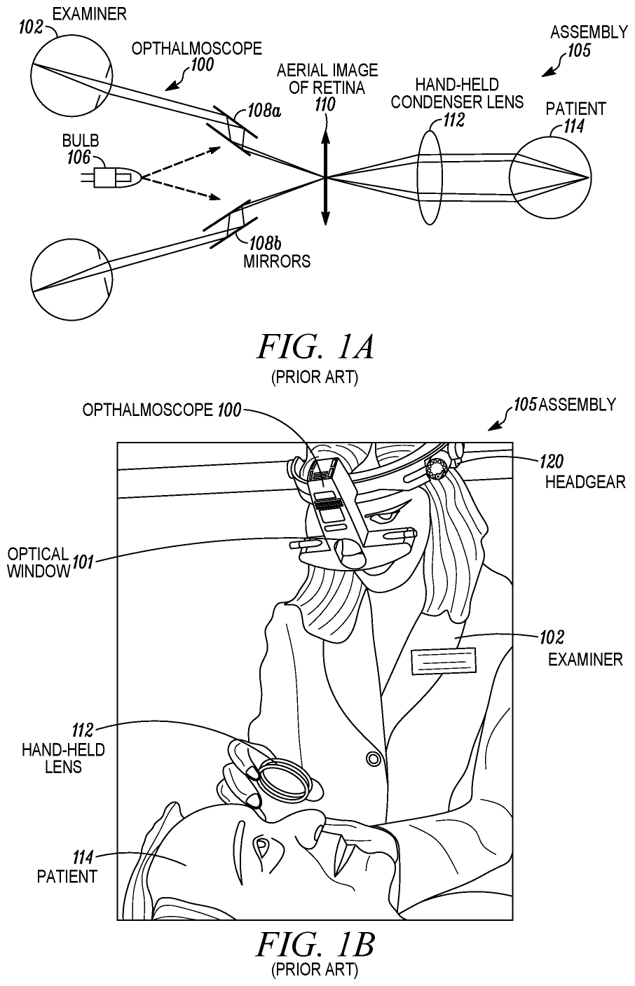

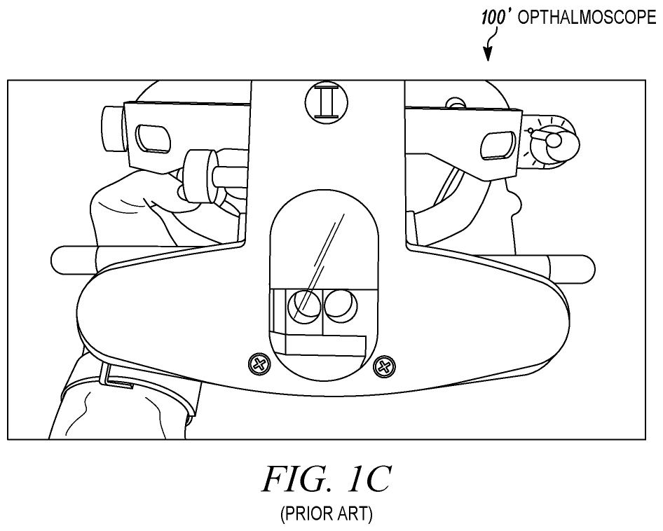

FIGS. 1A and 1B illustrate an example of an optical assembly 105 employing an indirect ophthalmoscope 100 to examine a patient 114. FIG. 1C shows a front view of a binocular indirect ophthalmoscope (e.g. showing the arrangement of two optical pupils near the bottom of FIG. 1C and the collimating optic of the illumination centered near the top of FIG. 1C). Binocular indirect ophthalmoscopy is one of the most common methods used by ophthalmologists to examine the retina of the patient 114. The optical assembly 105 includes headgear 120 and the binocular indirect ophthalmoscope 100 that consists of a binocular lens system with mirrors 108a, 108b, and a light source or bulb 106. The examiner 102 wears the ophthalmoscope 100 by positioning the headgear 120 so that the binocular lenses sit directly in front of their eyes. Mirrors 108a, 108b in the ophthalmoscope 100 split the light reflecting back toward the examiner 102 so that an image 110 of the retina may be presented to each eye of the examiner 102. The light source 106 is located in the center column within the ophthalmoscope 100 between the eyes of the examiner 102, just above the bridge of their nose. During binocular indirect ophthalmoscopy, the examiner 102 positions themselves at about arm's length from the eye of the patient 114. The examiner 102 then positions an indirect ophthalmoscopy lens 112 (otherwise known as a “hand-held condensing lens” or simply “condensing lens”) at a location (e.g. between about 20 mm and 80 mm) from the cornea of the patient 114. The hand-held condensing lens 112 is placed at this location such that the optical window 101 of the binocular indirect ophthalmoscope 100, the condensing lens 112, and the pupil of the patient 114, all are aligned along an optical axis.

A collimated light beam from the light source 106 is projected in an orthogonal direction out of the optical window 101 on the front of the binocular indirect ophthalmoscope 100. The light passes through the condensing lens 112 and the pupil of the patient 114 and illuminates the retina. The illuminated image of the fundus of the patient 114 is able to return along the same path, through the pupil, the condensing lens 112, and back to the optical window 101 of the binocular indirect ophthalmoscope 100.

A typical examination using the ophthalmoscope 100 takes anywhere from two to five minutes. Over this period of time the examiner 102 is in close proximity to the patient 114. Given the risk of viral outbreaks (e.g. COVID-19 and/or strains thereof), the inventors of the present invention identified an unmet need to provide a sanitary barrier of protection during this extended period of face to face contact (e.g. especially against viral or bacterial pathogens in a gaseous state such as aerosol and droplets). The inventors of the present invention recognized that conventional plastic face shields, affixed to the wearer by an elastic strap or flexible headband, are problematic to use in conjunction with a binocular indirect ophthalmoscope, which is already mounted by a headpiece. Additionally, the inventors of the present invention recognized that when a face shield is used in conjunction with a binocular indirect ophthalmoscope, reflections and distortions caused by the face shield negatively impact the image quality greatly.

Accordingly, embodiments of the proposed invention are presented herein, which provide an apparatus and method to securely attach a protective, sanitary barrier, to an optical assembly (e.g. binocular indirect ophthalmoscope), such that the usability and image quality of the original device are not affected. However, the method and apparatus of the present invention is not limited to the context of use with an ophthalmoscope and includes use with any device that features an optical assembly where an examiner may need a protective barrier.).

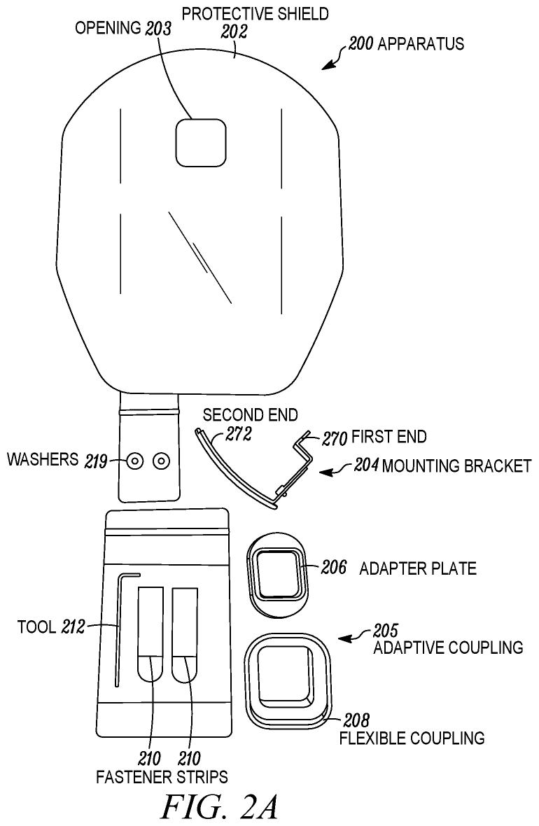

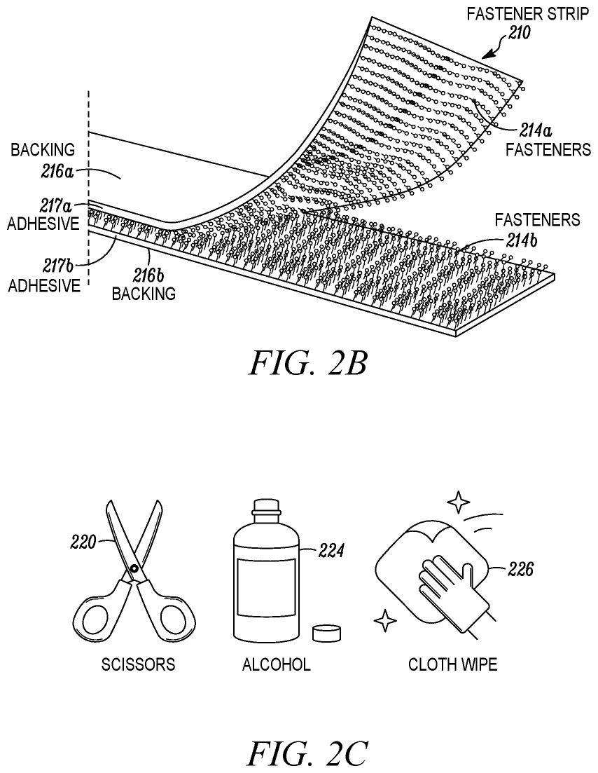

FIG. 2A is an image that illustrates an example of an apparatus 200 to provide a sanitary barrier for a user of an optical assembly, according to an embodiment. The apparatus 200 includes a protective shield 202 that is used to cover a face of the user. In an embodiment, the sanitary barrier includes the protective shield 202. In an example embodiment the protective shield 202 has an ergonomic tapered/trapezoidal shape allowing for unobstructed rotation of the user's head while also providing ideal frontal protection. In an example embodiment, the protective shield 202 is curved towards the user to provide additional lateral protection in addition to frontal protection.

In an embodiment, the protective shield 202 has dimensions that are sufficient to cover the face of the user (e.g. length of about 14 inches or in a range from about 10 inches to 18 inches and width of about 11 inches or in a range from about 9 inches to about 13 inches). In another embodiment, the protective shield 202 is made of material that is optically transparent (e.g. in the human visible spectrum). In an example embodiment, the protective shield 202 is made of clear flexible plastic material, e.g. Poly(methyl methacrylate) or PMMA; Polyethylene terephthalate or PET; Polycarbonate or PC. In an example embodiment, the protective shield 202 has a thickness in a range from about 0.010 inches to about 0.040 inches and/or in a range from about 0.005 inches to about 0.1 inches. In another embodiment the protective shield 202 has multiple layers of a removable plastic film which can be removed and disposed of, one at a time, in order to provide a more sanitary barrier. In an example embodiment, the protective shield 202 is curved in a concave shape, e.g. flexed towards the user. In an example embodiment, the radius of curvature of the concave shape is about 125 millimeters (mm) or in a range from about 80 mm to about 160 mm and/or in a range from about 40 mm to about 200 mm and/or in a range from about 20 mm to about 300 mm. However, the radius of curvature is not limited to any particular numerical range. In other embodiments, the radius of curvature is a compound radius, angled radius or flat. In another embodiment, the protective shield 202 has no curvature (e.g. planar protective shield) or is flexed in another direction.

In an embodiment, the protective shield 202 defines a window or opening 203. In one embodiment, the opening 203 is sized and/or positioned to provide an unimpeded optical path between a component of the optical assembly and a patient being examined by the user. In an example embodiment, the opening 203 is sized and/or positioned to provide an unimpeded optical path from the hand-held condensing lens 112 to the binocular indirect ophthalmoscope 100 and/or from the binocular indirect ophthalmoscope 100 to the user 102. In an embodiment, the protective shield 202 can be worn with a binocular indirect ophthalmoscope 100 and does not impede the user's 102 view (e.g. of the image 110) or restrict the user's ability to use the ophthalmoscope 100 in any other aspect. In one embodiment, the opening 203 may be cut in the protective shield 202. In an example embodiment, the opening 203 has a width of about 4 centimeters (cm) or in a range from about 3 cm to about 5 cm and a height of about 4 cm or in a range from about 3 cm to about 5 cm. In another example embodiment, the protective shield 202 defines the opening 203 at a location to be centered based on one or more criteria (e.g. an optical axis of the optical assembly such as an axis defined by the hand-held condensing lens 112, optical window 101 and eye of the patient 114). In another embodiment, the protective shield 202 does not define an opening 203. In yet another embodiment, the protective shield 202 defines multiple openings or windows.

In an embodiment, the apparatus 200 also includes a mounting fixture configured to attach the protective shield 202 to the headgear of the optical assembly (e.g. headgear 120 of the ophthalmoscope 100). In one embodiment, the mounting fixture of the apparatus 200 is different from a headgear fixture of the optical assembly that secures a housing with an optic to the headgear (e.g. headgear fixture of the ophthalmoscope 100 that secures the housing with the bulb 106 and mirrors 108 to the headgear 120). In this embodiment, the mounting fixture of the apparatus 200 is configured to attach the protective shield 202 to the headgear 120 independent of the headgear fixture of the ophthalmoscope 100 that attaches the ophthalmoscope 100 to the headgear 120. In another embodiment, the headgear fixture of the ophthalmoscope 100 secures the ophthalmoscope 100 to a hinge screw 122 of the headgear 120. In this embodiment, the mounting fixture of the apparatus 200 is configured to secure the protective shield 202 to the hinge screw 122, independent of the headgear fixture of the ophthalmoscope 100. In an embodiment, the mounting fixture of the apparatus 200 is a mounting bracket 204 that is configured to attach the protective shield 202 to the headgear of the optical assembly (e.g. headgear 120 of the ophthalmoscope 100). In an embodiment, the mounting bracket 204 includes a first end 270 that is configured to be secured to the headgear 120 and a second end 272 opposite to the first end 270 that is configured to be secured to (e.g. an inside surface) the protective shield 202. In an embodiment, the mounting bracket 204 is made of sheet metal (e.g. with a thickness of about 0.040″ or in a range from about 0.010″ to about 0.1″).

In an embodiment, the mounting bracket 204 positions the protective shield 202 in a desired location(s) relative to the optical assembly (e.g. binocular indirect ophthalmoscope 100). In another embodiment, the mounting bracket 204 also adapts to optical assemblies (e.g. binocular indirect ophthalmoscopes) of various makes and models. The protective shield 202 attaches to the optical assembly (e.g. binocular indirect ophthalmoscope 100) by the mounting bracket 204 that can be modified to accommodate existing hardware on the specific make and model of the optical assembly (e.g. binocular indirect ophthalmoscope 100). In one embodiment, the mounting bracket 204 provides the ability to position the protective shield 202 independently of the optical assembly 100 (e.g. binocular indirect ophthalmoscope 100), e.g. pivoting around the same hinge screw 122 common to most binocular indirect ophthalmoscope 100 designs. The inventors of the present invention recognized that this functionality allows users to store the optical assembly (e.g. ophthalmoscope 100) with the apparatus 200 remaining mounted and also allows users to lift the protective shield 202 when not in use.

In an embodiment, the apparatus 200 includes an adaptive coupling 205 with a first side configured to secure to the optical assembly and a second side opposite to the first side configured to engage the protective shield 202 (e.g. adjacent the opening 203). In one embodiment, the sanitary barrier of the apparatus 200 further includes the adaptive coupling 205. In an embodiment, the adaptive coupling 205 is configured to be mounted to the binocular indirect ophthalmoscope 100 and designed to interface with the protective shield 202. In an embodiment, the adaptive coupling 205 serves a purpose of sealing a gap between the ophthalmoscope 100 and the protective shield 202 (e.g. creating a first seal around the optical window 101 of the binocular indirect ophthalmoscope 100 and a second seal around the opening 203 of the protective shield 202) so that viral or bacterial pathogens within the gap cannot reach the user of the ophthalmoscope 100. Thus, in an embodiment, the protective shield 202 and the adaptive coupling 205 collectively form the sanitary barrier, where the adaptive coupling 205 extends the sanitary barrier to a same side of the protective shield 202 as the user, so to prevent viral or bacterial pathogens adjacent the opening 203 from reaching the user 102.

As shown in FIG. 2A, in one embodiment the adaptive coupling 205 includes an adapter plate 206 that is secured to the optical assembly (e.g. ophthalmoscope 100) and a flexible coupling 208 that engages the protective shield 202 (e.g. adjacent the opening 203). However, in other embodiments, the adaptive coupling 205 is just one component, where the adapter plate 206 and flexible coupling 208 are integrated as one-piece.

In one embodiment, the adaptive coupling 205 is attached to the ophthalmoscope 100 such that it extends forward towards the patient 114 to meet the inside surface (e.g. adjacent the opening 203) of the protective shield 202. In an embodiment, the adaptive coupling 205 is made of a close-celled foam and tube shaped following the perimeter of the optical window 101 on the ophthalmoscope 100. In another embodiment, the adaptive coupling 205 is made of flexible polymer (e.g. silicone or polyurethane). In an embodiment, the shape of the adaptive coupling 205, closely resembles corrugated bellows. In an embodiment, the adaptive coupling 205 has an adhesive backing that allows it to attach to the ophthalmoscope 100. In another embodiment the backing to the adaptive coupling 205 is adhesive backed hook and loop fabric, where the mating fabric is attached to the ophthalmoscope 100, allowing for removal and replacement of the adaptive coupling 205. In another embodiment a component of the adaptive coupling 205 (e.g. adapter plate 206) is a rigid coupler, fixed or removably attached to the ophthalmoscope 100 using double sided tape or Velcro™, and having a circumferential flange 240 with a barb feature, made to exchangeably receive a second component of the adaptive coupling 205 (e.g. flexible coupling 208). In yet another embodiment the patient facing surface of the adaptive coupling 205 (e.g. flexible coupling 208), tapers down to a thin, flexible edge that will more easily seal against the inside surface of the protective shield 202.

In an embodiment, the apparatus 200 of FIG. 2A also includes a pair of fasteners (e.g. fastener strips 210). In an embodiment, the fastener strips 210 are used to secure the mounting bracket 204 (e.g. second end 272) to the inside surface of the protective shield 202.

In an embodiment, the apparatus 200 of FIG. 2A also includes a tool 212 that is used to adjust a length of the mounting bracket 204 (e.g. to adjust an adjustment screw of the mounting bracket 204 that varies a length of the mounting bracket 204 between the hinge screw 122 and the protective shield 202).

In an embodiment, the apparatus 200 of FIG. 2A also includes washers 219 that are used to rotatably attach the mounting bracket 204 (e.g. first end 270) to the hinge screw 122.

Although FIG. 2A depicts one embodiment of the apparatus 200, the apparatus 200 need not include each component depicted in FIG. 2A and may exclude one or more of the washers 219, the tool 212 and the fastener strips 210. In some embodiments, the apparatus 200 includes the mounting bracket 204 and the protective shield 202. In other embodiments, the apparatus 200 includes the mounting bracket 204, the protective shield 202 and the washers 219.

FIG. 2B is an image that illustrates an example of a fastener strip 210 of the apparatus 200 of FIG. 2A, according to an embodiment. In an embodiment, the fastener strip 210 are snap-together fasteners with mushroom-shaped heads that interlock to hold two pieces pressed together. In an embodiment, the fastener strip 210 of FIG. 2B includes removable backing 216a, 216b on a top and bottom surface of the fastener strip 210. A respective adhesive layer 217a, 217b is inside the removable backing 216a, 216b such that upon peeling away the removable backing 216a, 216b, the adhesive layer 217a, 217b is exposed. The adhesive layer 217a, 217b conveniently permits each side of the fastener strip 210 to be adhered to a surface. In an embodiment, after peeling away the removable backing 216a, the adhesive 217a is affixed to a first surface (e.g. second end 272 of the mounting bracket 204) and after peeling away the removable backing 216b, the adhesive 217b is affixed to a second surface (e.g. inside surface of the protective shield 202 above the opening 203). In this embodiment, the fasteners 214a, 214b (e.g. hook and loop fasteners, mushroom-shaped heads that interlock, etc.) include any type of fastener known to one of ordinary skill in the art to secure the second end 272 of the mounting bracket 204 to the inside surface of the protective shield 202.

FIG. 2C is an image that illustrates an example of components used to install the apparatus 200 of FIG. 2A to the optical assembly, according to an embodiment. In an example embodiment, the components depicted in FIG. 2C are used to install the apparatus 200 to the ophthalmoscope 100. In an embodiment, the components include scissors 220, alcohol 224 and a cloth wipe 226. In an embodiment the components depicted in FIG. 2C are not components of the apparatus 200. In another embodiment, the components depicted in FIG. 2C are components of the apparatus 200.

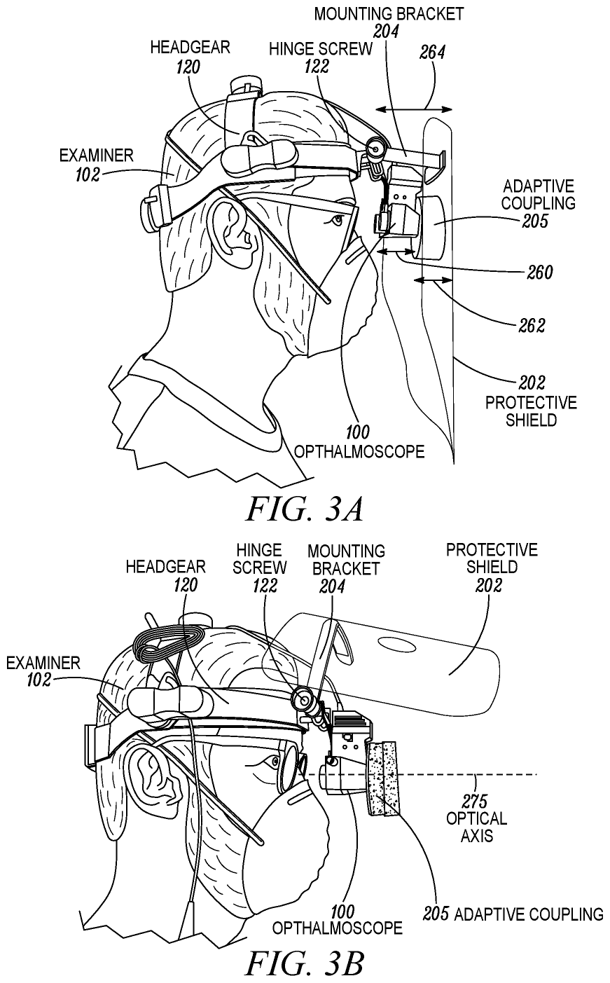

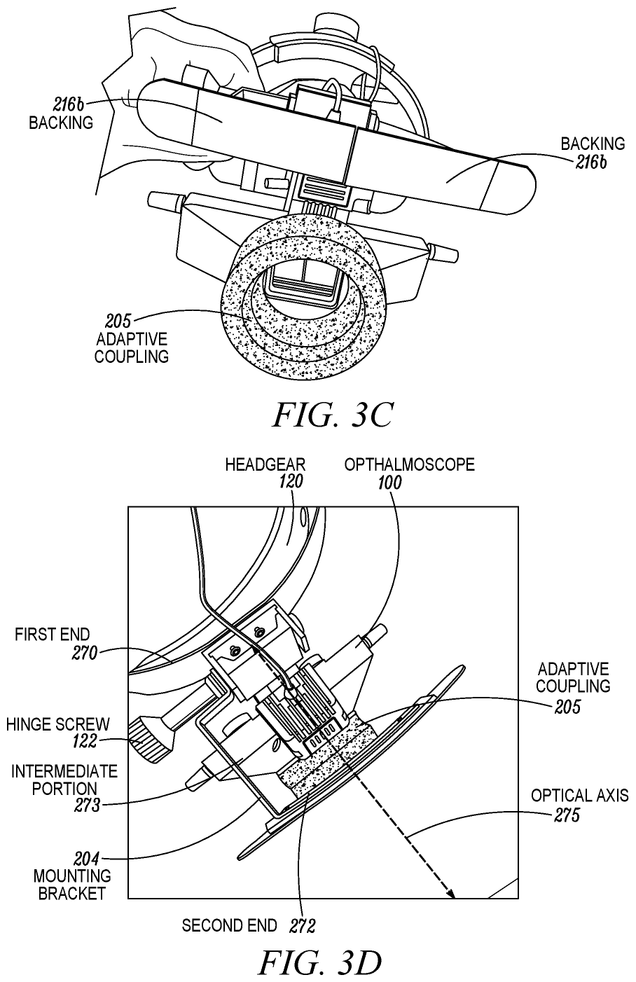

FIG. 3A is an image that illustrates an example of the apparatus 200 of FIG. 2A installed to an optical assembly and worn by a user in a first position, according to an embodiment. In an embodiment, the optical assembly is the ophthalmoscope 100 that is mounted with a headgear fixture to the hinge screw 122 of the headgear 120. In this embodiment, the protective shield 202 is mounted with a mounting fixture (e.g. mounting bracket 204) to the hinge screw 122 that is different from the headgear fixture. Thus, in an example embodiment, the protective shield 202 is mounted to the hinge screw 122 independent of the mounting of the ophthalmoscope 100 to the hinge screw 122. In another embodiment, the apparatus 200 of FIG. 3A is in a first position (e.g. lowered position) where the protective shield 202 is oriented about orthogonal (e.g. about 90 degrees or in a range of about 80 degrees to about 100 degrees) to an optical axis 275 (FIG. 3B) defined by one or more of the optical window 101, the hand-held condenser lens 122 and the retina of the patient 114 (not shown). As shown in FIG. 3A, the mounting bracket 204 extends from the hinge screw 122 to position the protective shield 202 between the patient 114 (not shown) and the ophthalmoscope 100. In another embodiment, the mounting bracket 204 extends from the hinge screw 122, to position the protective shield 202 between the examiner 102 and the ophthalmoscope 100.

As shown in FIG. 3A, a length 264 of the mounting bracket 204 is adjusted based on a width 260 of the ophthalmoscope 100 and a width 262 of the adaptive coupling 205 (e.g. the length 264 is adjusted to be about the same as a sum of the widths 260, 262). Thus, in an embodiment, the apparatus 200 can be adjusted to be installed on a variety of ophthalmoscopes 100 (e.g. that have varying width 260). Although the user in FIG. 3A is wearing a mask (e.g. personal protective equipment or PPE mask), the mask is not a component of the apparatus 200.

In another embodiment, FIG. 3A depicts that the adaptive coupling 205 is positioned between the ophthalmoscope 100 and the protective shield 202 and extends the sanitary barrier of the protective shield 202 to the same side of the protective shield 202 as the user, so that viral or bacterial pathogens which are adjacent to the opening 203 in the protective shield 202 do not reach the user (e.g. based on the first seal formed between the adaptive coupling 205 and the ophthalmoscope 100 and the second seal formed between the adaptive coupling 205 and the protective shield 202).

In another embodiment, the adaptive coupling 205 may be positioned within the opening and/or extend through the opening 203 towards the patient 114 (not shown) and engage an exterior surface of the protective shield 202. In yet another embodiment the adaptive coupling 205 may be positioned to extend backwards, upwards, downwards, sideways or any combination of the previous to meet the protective shield 202.

FIG. 3B is an image that illustrates an example of the installed apparatus 200 of FIG. 3A worn by a user in a second position, according to an embodiment. In an embodiment, the mounting bracket 204 rotatably mounts the protective shield 202 to the hinge screw 122, independent of the headgear fixture that mounts the ophthalmoscope 100 to the hinge screw 122. Thus, as depicted in FIG. 3B, the user can rotate the protective shield 202 from the lowered position (FIG. 3A) to a raised position (FIG. 3B). Although the ophthalmoscope 100 is rotatably mounted to the hinge screw 122, the ophthalmoscope 100 remains in the same positions in FIGS. 3A and 3B and thus the apparatus 200 advantageously permits the protective shield 202 to be rotated about the hinge screw 122 independent of the ophthalmoscope 100. Although FIGS. 3A and 3B depict that the protective shield 202 and the ophthalmoscope 100 are each rotatable about the hinge screw 122, in other embodiments, the protective shield 202 and ophthalmoscope 100 do not share the same pivot point (e.g. the protective shield 202 is rotatably attached to a component of the optical assembly other than the hinge screw 122 or the protective shield 202 is non-rotatably attached to the optical assembly).

FIG. 3C is an image that illustrates an example of the mounting bracket 204 and adaptive coupling 205 of the apparatus 200 of FIG. 2A to engage the protective shield, according to an embodiment. In an embodiment, the backing 216b of two fastener strips 210 are depicted along the second end 272 of the mounting bracket 204, after the backing 216a of the two strips 210 was removed and the adhesive 217a of both strips 210 affixed along the second end 272 of the mounting bracket 204. In an embodiment, after peeling away the backing 216b of the two strips 210 shown in FIG. 3C, the adhesive 217b of the two strips 210 can be affixed along the inside surface of the protective shield 202 (e.g. above the opening 203) to secure the mounting bracket 204 to the protective shield 202.

FIG. 3C also depicts the adaptive coupling 205, which forms a circular edge that is sized and configured to engage the inside surface of the protective shield 202 adjacent to the opening 203. In one embodiment, the edge of the adaptive coupling 205 is sized and configured to enclose the opening 203 and form a seal with the protective shield 202 adjacent to the opening 203 (e.g. such that viral or bacterial pathogens cannot pass between the protective shield 202 and the adaptive coupling 205 and reach the user 102). In other embodiments, the edge of the adaptive coupling 205 is configured to be positioned within (e.g. frictional fit) the opening 203 so to provide the seal with the protective shield 202.

FIG. 3D is an image that illustrates an example of a top view of the installed apparatus 200 of FIG. 3A, according to an embodiment. In an embodiment, the first end 270 of the mounting bracket 204 is positioned such that the hinge screw 122 passes through an opening (not shown) in the first end 270 to rotatably attach the mounting bracket 204 (and protective shield 202) to the hinge screw 122. Additionally, FIG. 3D depicts an intermediate portion 273 of the mounting bracket 204 between the first end 270 and the second end 272, where the intermediate portion 273 is oriented parallel to the optical axis 275 (when the apparatus is in the lowered position of FIG. 3A). In an embodiment, the length of the mounting bracket 204 (parallel to the optical axis 275) is adjusted by varying a length of the intermediate portion 273 (e.g. with an adjustment screw 250 and two bracket portions 252a and 252b) along the optical axis 275. In yet another embodiment, FIG. 3D depicts that the length 264 of the intermediate portion 273 is adjusted based on a sum of the width 260 of the ophthalmoscope 100 and the width 262 of the adaptive coupling 205 (along the optical axis 275).

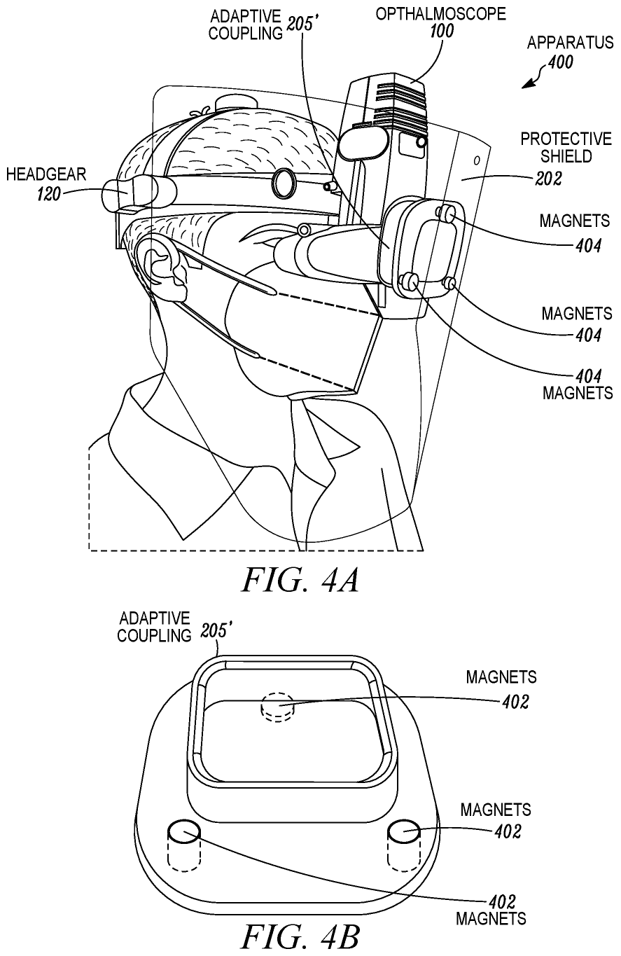

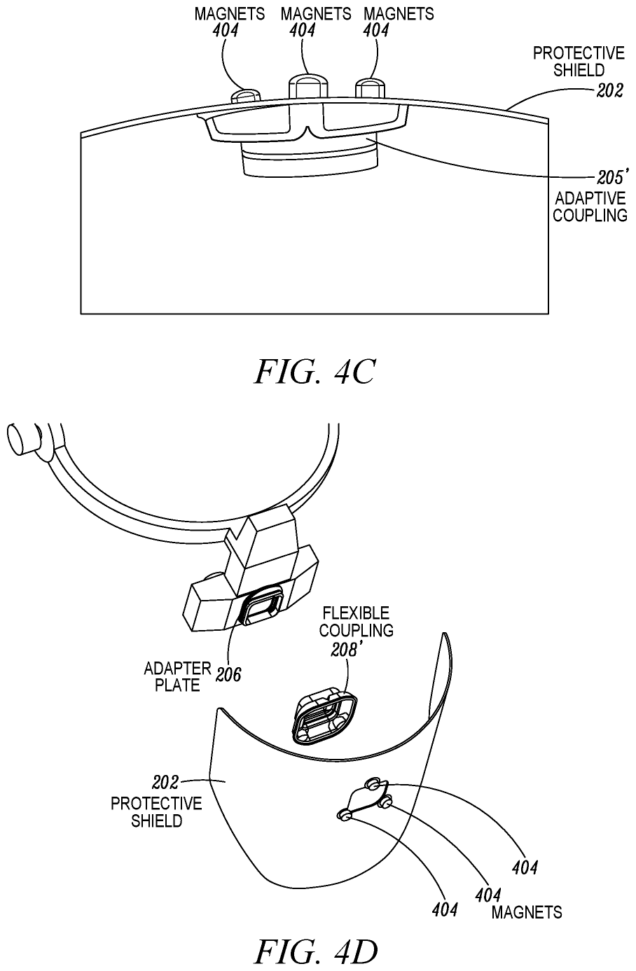

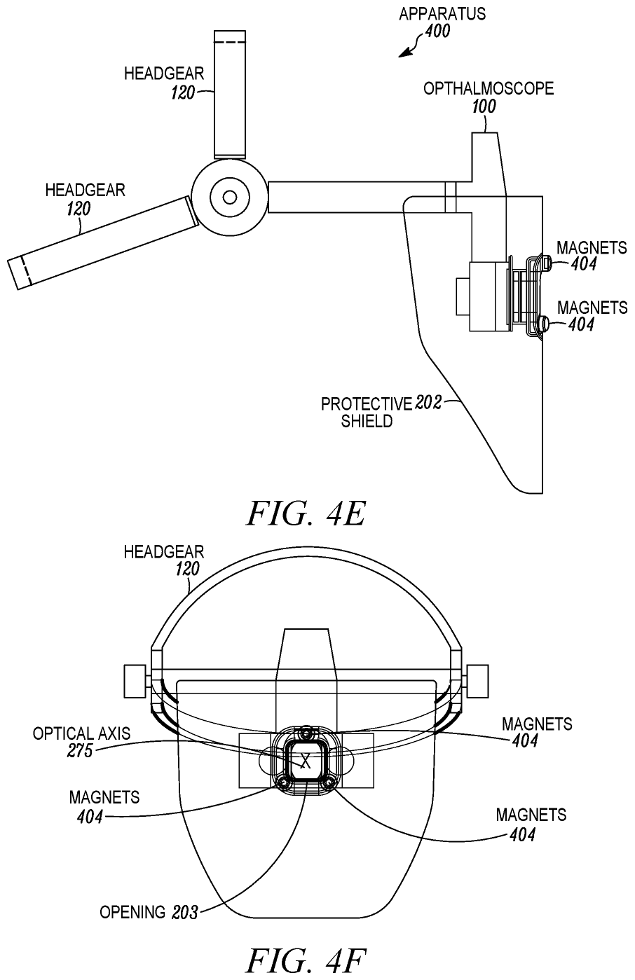

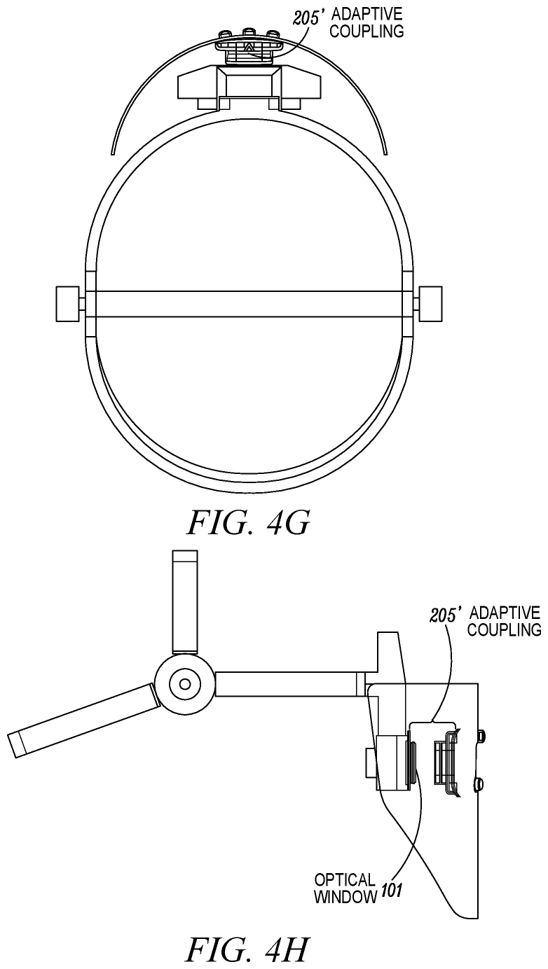

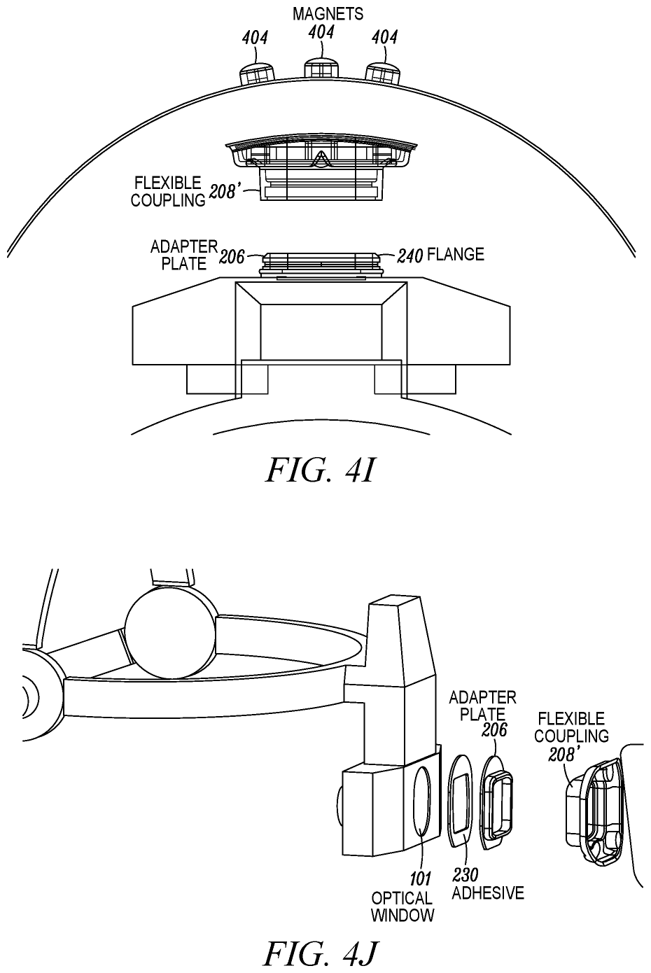

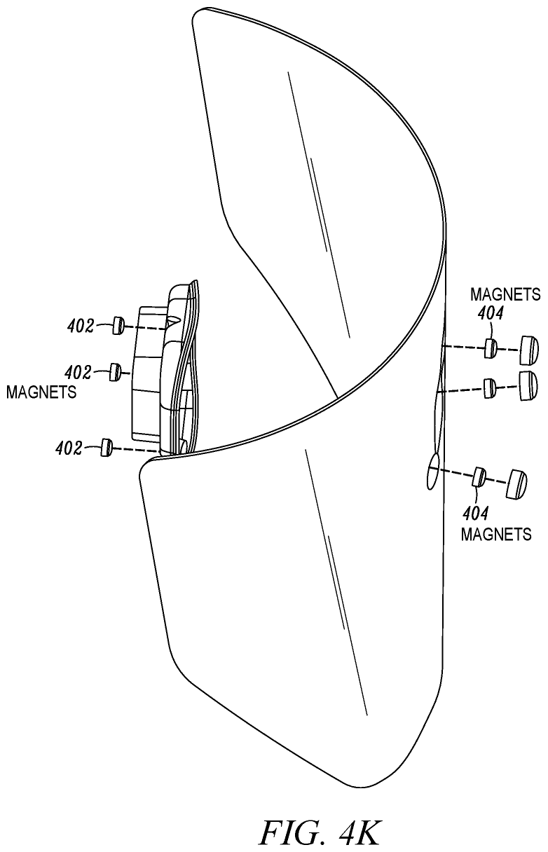

FIG. 4A is an image that illustrates an example of a front perspective view of an apparatus 400 to provide a sanitary barrier for a user of an optical assembly, according to an embodiment. In an embodiment, the apparatus 400 is similar to the apparatus 200 previously discussed, with the exception of the features discussed herein. Unlike the apparatus 200 with the mounting fixture (e.g. mounting bracket 204) that mounts the protective shield 202 to the headgear 120, the apparatus 400 features a mounting fixture (e.g. adaptive coupling 205′) that mounts the protective shield 202 to the housing of the optical assembly (e.g. housing of the ophthalmoscope 100). Additionally, as with the apparatus 200, the adaptive coupling 205′ provides an airtight seal in the gap between the housing of the ophthalmoscope 100 and the protective shield 202, such that the sanitary barrier further includes the adaptive coupling 205′. In an example embodiment, the mounting bracket 204, the fastener strips 210, the tool 212 and/or the washers 219 of the apparatus 100 are not included in the apparatus 400, since they are utilized to mount the protective shield 202 to the headgear 120.

As shown in FIG. 4A, in one embodiment the protective shield 202 is mounted to the housing of the optical assembly (e.g. housing of the ophthalmoscope 100) with the mounting fixture (e.g. adaptive coupling 205′). In one embodiment, the mounting fixture (e.g. adaptive coupling 205′) is configured with a centering feature such that upon mounting the protective shield 202 to the housing of the optical assembly, the opening 203 is aligned with the optical axis 275 (FIG. 3B) of the optical assembly. This advantageously ensures that the opening 203 is aligned and centered with the optical axis 275 of the optical assembly, to provide an unobstructed line of sight through the protective shield 202 to the optical assembly.

In one embodiment, the mounting fixture is the adaptive coupling 205′, which is similar to the adaptive coupling 205 of the apparatus 200, with the exception of the features discussed herein. In one embodiment, a magnetic arrangement (e.g. one or more magnets), or another temporary binding method, connect the adaptive coupling 205′ to the protective shield 202 and help to form a complete seal around the opening 203. In other embodiments, other temporary binding methods employed include one or more of an adhesive, suction cup style fasteners, hook and loop fabric, snap clips, or other temporary fasteners commonly used by those skilled in the art. This connection will advantageously also keep the shield 202 attached to the ophthalmoscope 100 but continue to allow for easy removal, storage, or cleaning. In an embodiment, the adaptive coupling 205′ is injected molded with a TPE (thermoplastic elastomer), such as a SEBS (Styrene-Ethylene-Butylene-Styrene). As shown in FIG. 4A, in one embodiment, the magnetic arrangement includes a plurality of magnets 402 arranged around a side of the adaptive coupling 205′ that engages the protective shield 202. In one example embodiment, three magnets 402 are arranged around the side of the adaptive coupling 205′ in a triangular arrangement. In an embodiment, the magnets 402 are configured to attract a plurality of magnets 404 (not shown in FIG. 4A) arranged around the opening 203 of the protective shield 202. In this embodiment, attraction between the magnets 402 on the adaptive coupling 205′ and the magnets 404 on the protective shield 202 mounts the protective shield 202 to the housing of the optical assembly and presses the protective shield 202 against the adaptive coupling 205′ to form the seal between optical assembly and the protective shield 202. Additionally, the adaptive coupling 205′ provides a self-centering ability that centers the opening 203 along the optical axis 275, when the magnets 402 on the adaptive coupling 205′ attract the magnets 404 on the protective shield 202. Although three magnets 402 in a triangular arrangement are depicted in FIG. 4A, in other embodiments less or more than three magnets 402 are provided and/or in a different arrangement than the triangular arrangement, provided that the magnets 402 are configured to attract a corresponding magnetic arrangement around the opening 203 on the protective shield 202. In yet another embodiment, the magnets 404 on the protective shield 202 are configured to attract a corresponding magnetic arrangement on the adaptive coupling 205′. In another example embodiment, magnets are only arranged on one side (e.g. one of the adaptive coupling 205′ or protective shield 202) and the other side (e.g. the other of the adaptive coupling 205′ or protective shield 202) includes a magnetic arrangement featuring a ferromagnetic material (e.g, iron, nickel, cobalt, etc.) that attracts the magnets.

FIG. 4B is an image that illustrates an example of a side perspective view of the adaptive coupling 205′ of the apparatus 400 of FIG. 4A, according to an embodiment. In an embodiment, the three magnets 402 are depicted in the triangular arrangement. In an example embodiments, a plurality of cavities are formed in the adaptive coupling 205′ to receive the respective plurality of magnets 402. In an example embodiment, the magnets 402 are embedded within cavities within the flexible coupling 208′ of the adaptive coupling 205′, which is similar to the flexible coupling 208 other than the embedded magnets 402. In another example embodiment, the magnets 402 are cylindrical magnets and thus the cavities take a cylindrical shape. However, the magnets are not limited to any particular shape. As shown in FIG. 4B, in one embodiment a magnet 402 is positioned on the adaptive coupling 205′ at each corner of a base of the adaptive coupling 205′ and another magnet 402 is positioned near a center of a top of the adaptive coupling 205′. In another embodiment, a magnet 402 is positioned on the adaptive coupling 205′ at each corner along a top of the adaptive coupling 205′ and another magnet 402 is arranged near a center of a base of the adaptive coupling 205′. In still another embodiment, a magnet 402 can be positioned at each corner of the adaptive coupling 205′.

FIG. 4C is an image that illustrates an example of a top view of an adaptive coupling 205′ secured to the sanitary barrier of the apparatus 400 of FIG. 4A, according to an embodiment. In an embodiment, FIG. 4C shows a top view of a portion the adaptive coupling 205′ (e.g. flexible coupling 208′) attached to the protective shield 202. In one embodiment, as shown in FIG. 4C, a complete seal is formed and no gaps are present between the adaptive coupling 205′ and the protective shield 202. In one embodiment, magnets 404 are permanently attached to the front side of the protective shield 202, and the attraction between these magnets 404 and the magnets 402 is what creates the force to form the seal and keep the protective shield 202 attached to the adaptive coupling 205′.

In one embodiment, as shown in FIG. 4C, the magnets 404 are attached to the front side of the protective shield 202 but in other embodiments the magnets 404 can be attached to the rear side of the protective shield 202 (facing the ophthalmoscope 100) and can be configured to be received within the cavities on the adaptive coupling 205′. In an embodiment, the adaptive coupling 205′ provides a self-centering ability that centers the opening 203 along the optical axis 275, when the magnets 402 on the adaptive coupling 205′ attract the magnets 404 on the protective shield 202. In some embodiments, the self-centering feature that centers the opening 203 along the optical axis 275 is done by magnetic attraction between the magnets 402, 404. In other embodiments, the self-centering feature that centers the opening 203 along the optical axis 275 is done by magnetic attraction between the magnets 402, 404 in tandem with one or more protrusions on one of the protective shield 202 and the adaptive coupling 205′ configured to be received within one or more respective cavities (e.g. cavities that house one or more magnets) on another of the protective shield 202 and the adaptive coupling 205′. In yet another embodiment, the magnets 402, 402 could be affixed directly (e.g. using an adhesive, such as glue) to the either the adaptive coupling 205′ or the protective shield 202. In an embodiment, the magnets 404 permanently attached to the protective shield 202 have the same quantity and arrangement as the magnets 402 attached to the adaptive coupling 205′.

FIG. 4D is an image that illustrates an example of an exploded view of the apparatus 400 of FIG. 4A, according to an embodiment. FIG. 4D shows two portions of the adaptive coupling 205′, the adapter plate 206 and the flexible coupling 208′. In an embodiment, a rigid portion (e.g. adapter plate 206) of the adaptive coupling 205′ contains a circumferential flange 240 (FIG. 41) that can be securely seated within a more flexible portion (e.g. flexible coupling 208′) of the adaptive coupling 205′. In another embodiment, the rigid portion (e.g. adapter plate 206), also possesses an adhesive backing 230 (FIG. 4J) that enables the adapter plate 206 of the adaptive coupling 205′ to attach to the housing of the optical assembly (e.g. ophthalmoscope 100). In this embodiment, together the adapter plate 206, the flexible coupling 208′, the adhesive backing 230, and the coupling magnets 402, all form the adaptive coupling 205′. In an embodiment, the adaptive coupling 205′ is secured to the housing of the optical assembly, in a similar manner as the adaptive coupling 205 of the apparatus 200.

FIG. 4E is an image that illustrates an example of a side view of the apparatus 400 of FIG. 4A, according to an embodiment. In an embodiment, FIG. 4E depicts the magnets 404 attached to the front side of the protective shield 202. FIG. 4F is an image that illustrates an example of a front view of the apparatus 400 of FIG. 4A, according to an embodiment. In an embodiment, FIG. 4F depicts the self-centering ability of the adaptive coupling 205′, where the opening 203 is centered along the optical axis 275, based on the magnetic attraction between the magnets 402, 404.

FIG. 4G is an image that illustrates an example of a top view of the apparatus 400 of FIG. 4A, according to an embodiment. In an embodiment, as shown in FIG. 4G, the protective shield 202 is curved (e.g. concave) towards the user to provide additional lateral protection in addition to frontal protection. FIGS. 4H through 4K are images that illustrate examples of various exploded views of the apparatus of FIG. 4A, according to an embodiment.

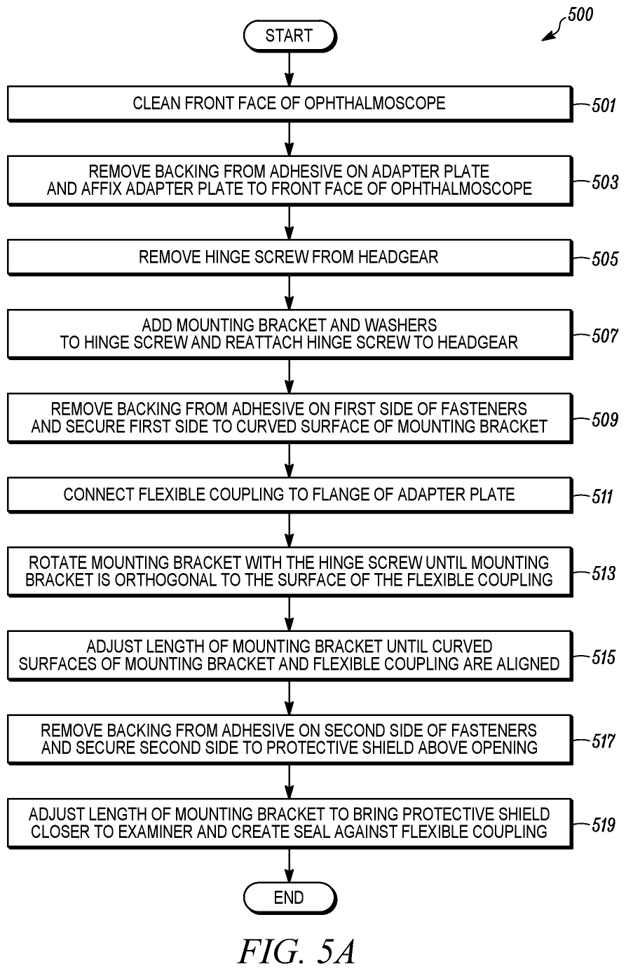

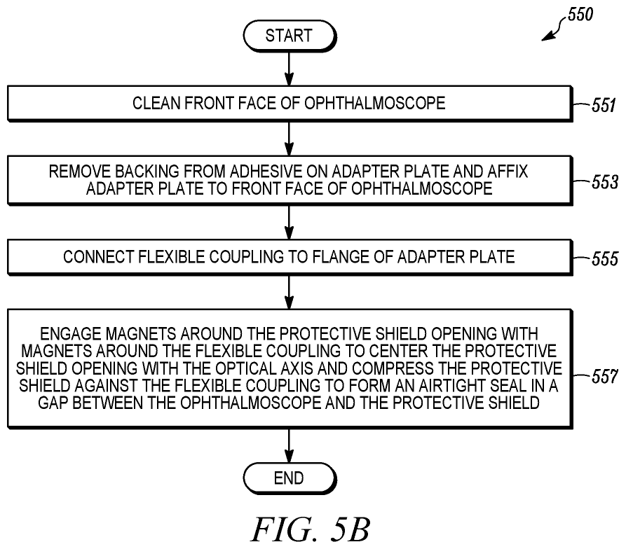

FIG. 5A is a flow diagram that illustrates an example of a method 500 for installing the apparatus 200 of FIG. 2A to an optical assembly (e.g. ophthalmoscope 100), according to an embodiment. FIG. 5B is a flow diagram that illustrates an example of a method 550 for installing the apparatus 400 of FIG. 4A to an optical assembly (e.g. ophthalmoscope 100), according to an embodiment. Although steps are depicted in FIGS. 5A and 5B as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In an embodiment, the method 500 of FIG. 5A presumes that the user already has an optical assembly (e.g. ophthalmoscope 100) to which the user wants to install the assembly 200 to enhance the use of the optical assembly (e.g. provide a sanitary barrier). In some embodiments, certain components (e.g. scissors 220, alcohol 224 and/or cloth wipe 226 of FIG. 2C) that are not part of the apparatus 200 are used in performing the method 500. However, in an embodiment, these components are not a part of the apparatus 200. In other embodiments, these components are a part of the apparatus 200.

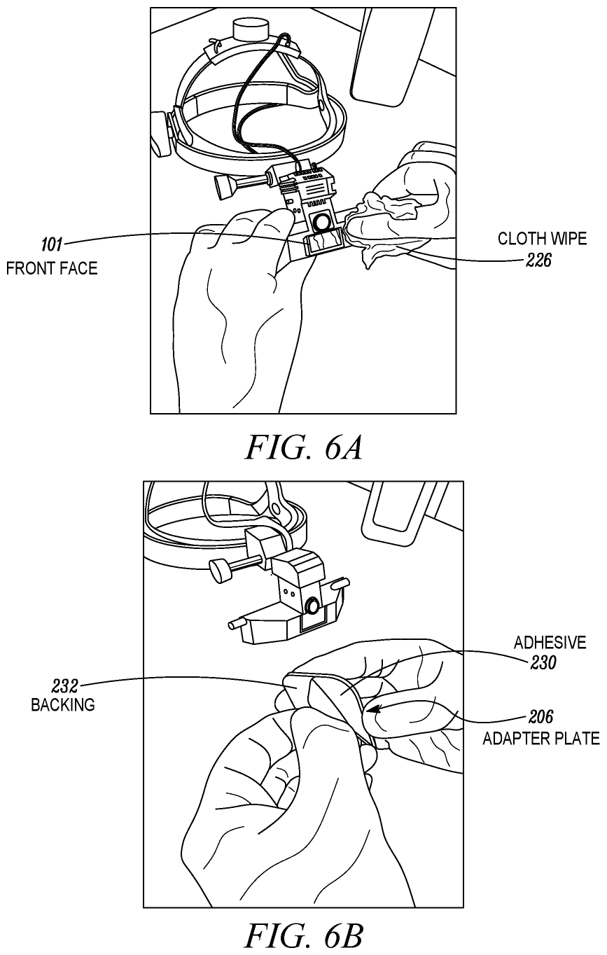

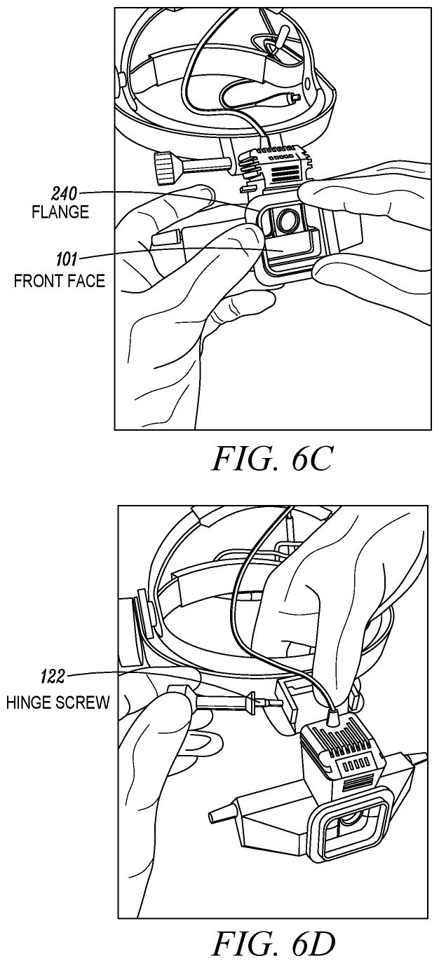

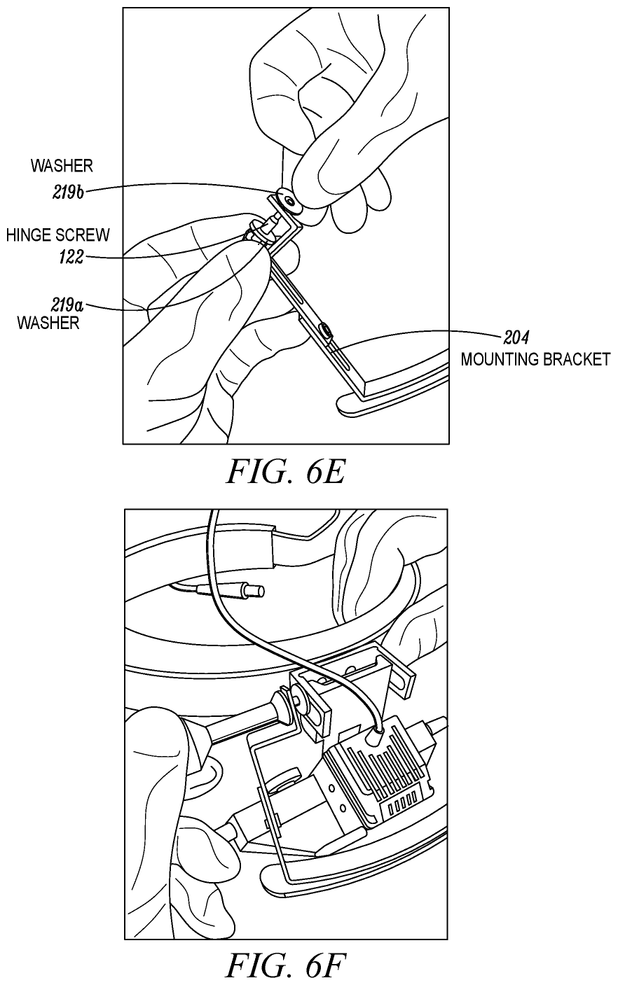

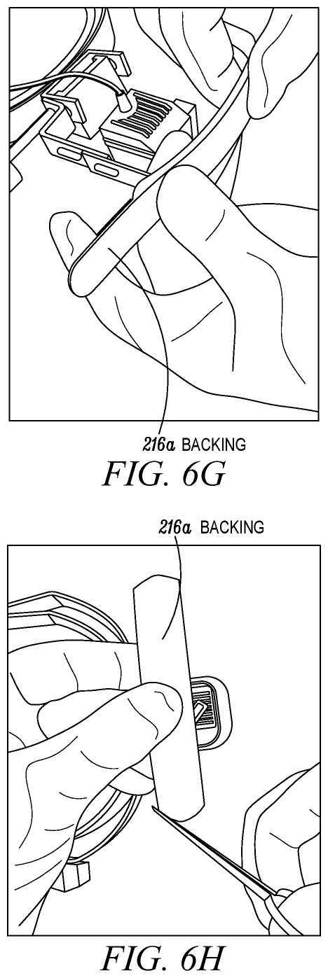

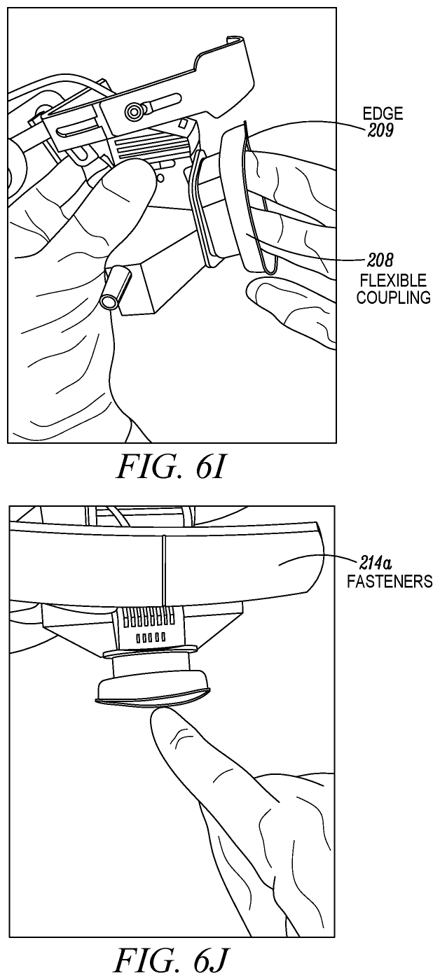

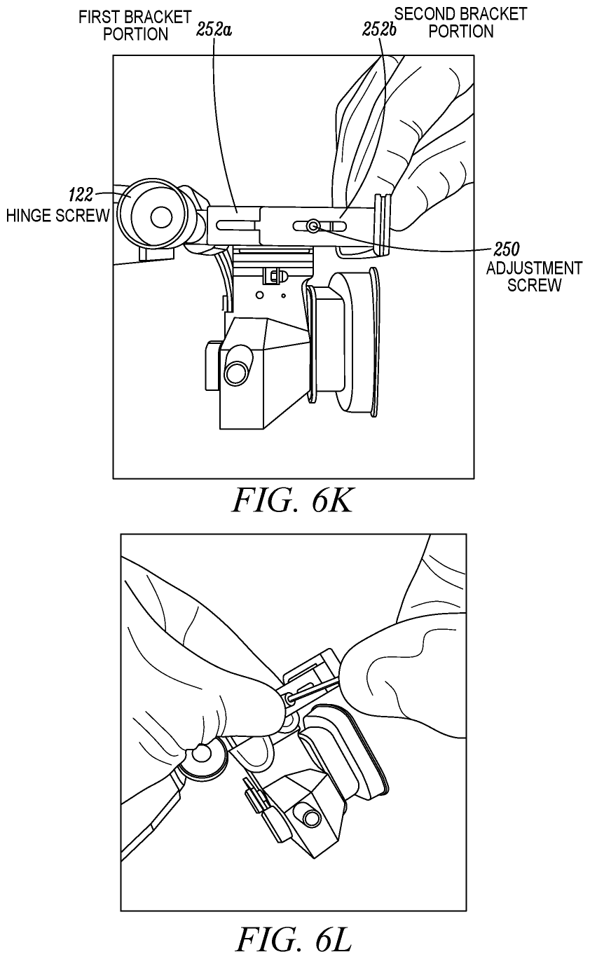

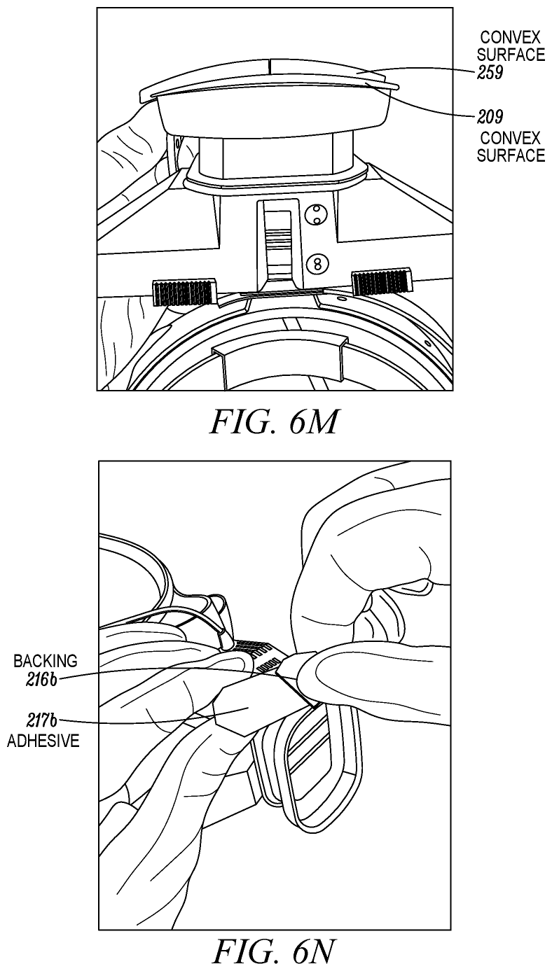

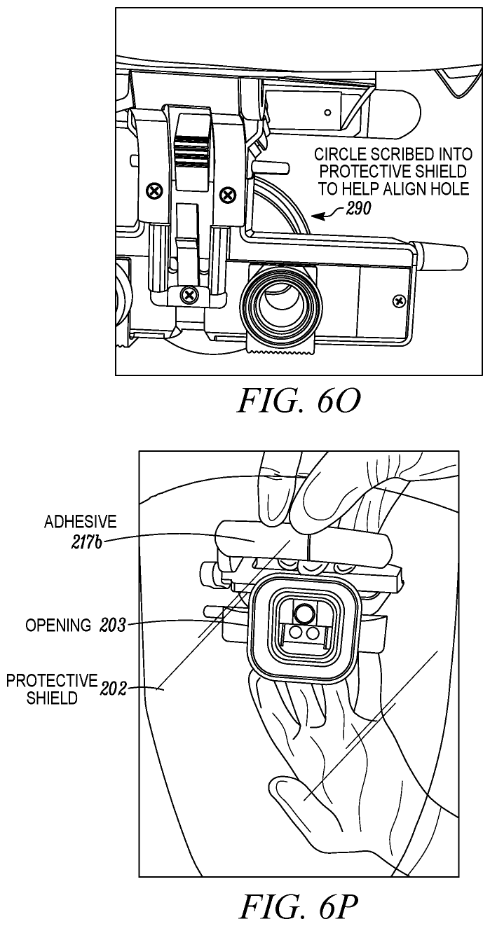

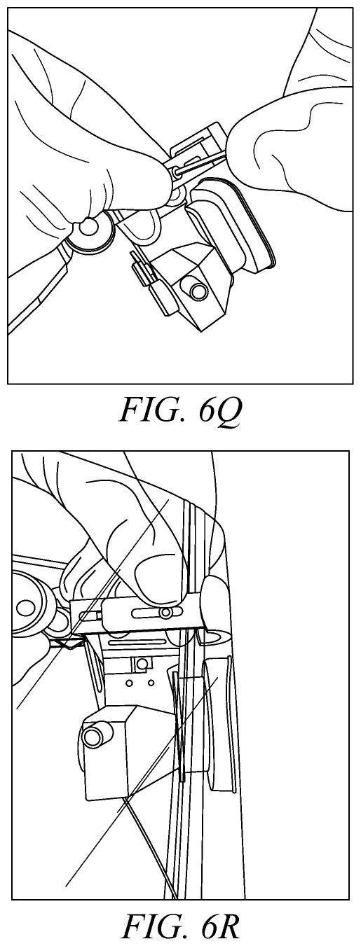

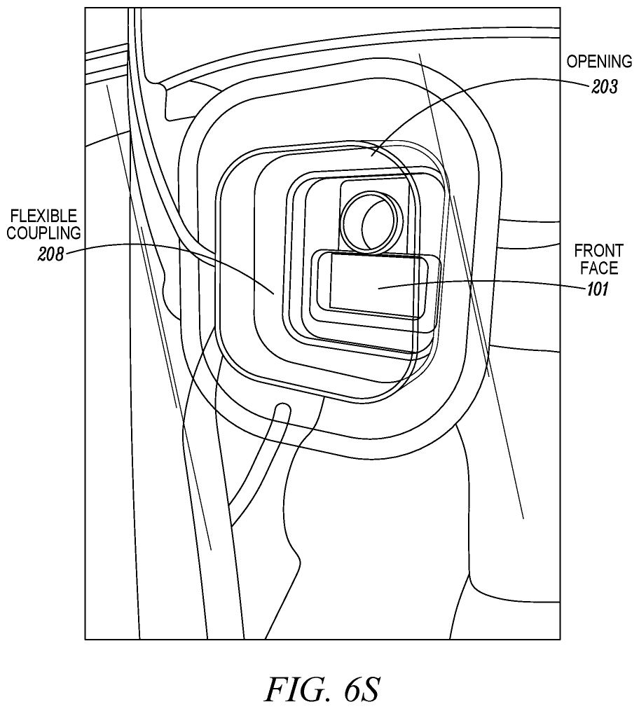

In an embodiment, in step 501 a face of the optical assembly is cleaned. In one embodiment, in step 501 the front face of the ophthalmoscope 100 is cleaned. FIG. 6A depicts one embodiment of performing step 501. In an example embodiment, in step 501 the cloth wipe 226 is dampened with a cleaning agent (e.g. alcohol 224, such as isopropanol) and the front face is wiped with the dampened cloth wipe 226. As appreciated by one of ordinary skill in the art, the front face of the ophthalmoscope 100 is a part of the housing of the ophthalmoscope 100 that is in the same plane as the optical window 101 (e.g. optically transparent portion of the housing, distinct from the non-transparent front face).

In an embodiment, in step 503 the adaptive coupling 205 is secured to the optical assembly. In one embodiment, in step 503 the adapter plate 206 of the adaptive coupling 205 is secured to the housing (e.g. that includes the mirrors 108 and light source 106) of the ophthalmoscope 100. FIGS. 6B and 6C depicts one embodiment of performing step 503. In one embodiment, as shown in FIG. 6B, in step 503 a backing 232 is removed from a first side of the adapter plate 206, that exposes an adhesive 230. In this embodiment, as shown in FIG. 6C, to affix the adapter plate 206 to the housing of the ophthalmoscope 100, the adapter plate 206 is centered over the optical window 101 and the adhesive 230 firmly applied to the front face of the ophthalmoscope 100. Although the above embodiment of step 503 discusses the use of an adhesive and removable backing to secure the adapter plate 206 to the ophthalmoscope 100, in other embodiments any fastener or adhesive appreciated by one of ordinary skill in the art can be used to secure the adapter plate 206 to the housing of the ophthalmoscope 100. As shown in FIG. 6C, in one embodiment, after attaching a first side of the adapter plate 206 to the ophthalmoscope 100, a second side opposite to the first side that includes a flange 240 is provided (e.g. to secure the flexible coupling 208 to the adapter plate 206 in step 511).

In an embodiment, in step 505 the hinge screw 122 is removed from the headgear 120 separating the headgear 120 from the ophthalmoscope 100. FIG. 6D depicts one embodiment of performing step 505. In one example embodiment, a tool (e.g. thumb screw) is employed in step 505.

In an embodiment, in step 507 the mounting bracket 204 is rotatably attached to the hinge screw 122 (e.g. so that the protective shield 202 and mounting bracket 204 are rotatably connected to the hinge screw 122). FIGS. 6E and 6F depict one embodiment of performing step 507. In one embodiment, after removing the hinge screw 122 in step 505, a first washer 219a is placed on the hinge screw 122. After placing the first washer 219a on the hinge screw 122, the hinge screw 122 is passed through an opening defined by the first end 270 of the mounting bracket 204. In an embodiment, after passing the hinge screw 122 through the opening in the mounting bracket 204, a second washer 219b is placed on the hinge screw 122. In an embodiment, after passing the two washers 219a, 219b and the mounting bracket 204 through the hinge screw 122 (e.g. where the washers 219a, 219b are on opposite sides of the mounting bracket 204), the hinge screw 122 is tightened, reattaching the headgear 120 to the ophthalmoscope 100, with the new addition of the mounting bracket 204 and the two washers 219a, 219b. In one embodiment, the washers 219 are made of a low friction material, such as PTFE, PE, PA. Although FIGS. 6E and 6F depict one embodiment of rotatably attaching the mounting bracket 204 to the hinge screw 122, in other embodiments the mounting bracket is rotatably attached to the ophthalmoscope 100 at a location other than the hinge screw 122 or is non-rotatably attached to a component of the ophthalmoscope 100. Additionally, the embodiment of FIGS. 6E and 6F merely depicts one structural arrangement of rotatably attaching the mounting bracket 204 to the hinge screw 122 and any known method or structural arrangement known to one skilled in the art for rotatably attaching the mounting bracket 204 to the hinge screw 122 can be employed.

In an embodiment, in step 509 the backing 216a is removed from a first side of the fastener strip 210 and the first side of the fastener strip 210 is affixed to the second end 272 of the mounting bracket 204. FIGS. 6G and 6H depict one embodiment of performing step 509. In an embodiment, the fastener strip 210 is first aligned with the second end 272 of the mounting bracket 204. Based on this alignment, the scissors 220 (FIG. 2C) may be used to cut portions of the fastener strip 210 (FIG. 6H) that extend beyond the second end 272. After cutting the fastener strip 210 so that the fastener strip 210 lines up with the second end 272, the backing 216a is removed and the exposed adhesive 217a is used to secure the fastener strip 210 along the second end 272. In some embodiments, the apparatus 200 is provided with the fastener strip 210 already secured to the second end 272 and thus in this embodiment step 509 is omitted. Additionally, FIGS. 6G and 6H merely depict one type of fastener (e.g. fastener strip 210) that can be secured to the mounting bracket 204. In other embodiments, any type of fastener that is well known in the art (e.g. hook and loop fasteners, Velcro®, adhesive, etc.) different from the fastener strip 210 can be secured to the mounting bracket 204 in step 509.

In an embodiment, in step 511 the flexible coupling 208 is attached to the adapter plate 206 that was secured to the ophthalmoscope 100 in step 503. FIGS. 6I and 6J depict one embodiment of performing step 511. As shown in FIG. 6I, in one embodiment, the flexible coupling 208 includes a first side (left side in FIG. 6I) with a first outer diameter and a second side (right side in FIG. 6I) with a second outer diameter that is larger than the first outer diameter. In an example embodiment, the first outer diameter is about 40 mm or in a range from about 30 mm to about 50 mm and the second outer diameter is about 80 mm or in a range from about 60 mm to about 100 mm. As shown in FIG. 6C, in step 503 a first side of the adapter plate 206 is secured to the ophthalmoscope 100 and a second side opposite to the first side features a flange 240 that extends around a perimeter of the adapter plate 206. In an embodiment, in step 511 the first side of the flexible coupling 208 is secured to the flange 240. In an example embodiment, in step 511 an inner surface of the first side of the flexible coupling 208 frictionally engages the flange 240 and forms an air-tight seal (e.g. the internal wall of the first side of the flexible coupling 208 fits over the flange 240 with a tight frictional fit).

In another embodiment, in step 511 after securing the flexible coupling 208 to the adapter plate 206, as shown in FIG. 6J a curvature of the top and bottom portions of the second side of the flexible coupling 208 matches the curvature of the second end 272 of the mounting bracket 204. In an example embodiment, the curvature of the second end of the flexible coupling 208 and the curvature of the second end 272 are convex (e.g. with a radius of curvature in a range from about 80 mm to about 200 mm and/or in a range from about 20 mm to about 300 mm). In another example embodiment, the curvature of the second side of the flexible coupling 208 and/or the curvature of the second end 272 of the mounting bracket 204 are based on (e.g. about equal to) the curvature of the inside surface of the protective shield 202. In another embodiment, the protective shield 202 is flat and conforms to the curvature of the flexible coupling 208 and mounting bracket 204 once the protective shield 202 is mounted to the flexible coupling 208 and mounting bracket 204.

In an embodiment, in step 513 a rotational position of the mounting bracket 204 about the hinge screw 122 is adjusted until the mounting bracket 204 is about orthogonal (e.g. about 90 degrees or in a range from about 80 degrees to about 100 degrees) to the second side of the flexible coupling 208. FIG. 6K depicts one embodiment of performing step 513. As shown in FIG. 6K, in one embodiment, in step 513 the mounting bracket 204 is rotated relative to the hinge screw 122 until the orientation of the mounting bracket 204 (e.g. intermediate portion 273) is aligned about orthogonal to the second side of the flexible coupling 208.

In an embodiment, in step 515 after aligning the mounting bracket 204 in step 513 to be about orthogonal to the second side of the flexible coupling 208, in step 515 the length of the mounting bracket 204 is adjusted until the second end 272 of the mounting bracket 270 is aligned with the convex surface of the second end of the flexible coupling 208. FIGS. 6K through 6M depict one embodiment of performing step 515. In one embodiment, in step 515 an adjustment screw 250 is loosened (e.g. using tool 212) so to be able to move a second bracket portion 252b relative to a first bracket portion 252a and consequently adjust a length of the mounting bracket 204 (in a direction of the optical axis 275). In some embodiments, step 515 involves adjusting the length 264 (FIG. 3A) based on the width 260 of the ophthalmoscope 100 and the width 262 of the adaptive coupling 206. In an example embodiment, in step 515 the length 264 is adjusted based on the sum of the width 260 and 262. In an embodiment, step 515 advantageously permits the apparatus 200 to be installed on various ophthalmoscopes with varying width 260. After the second end 272 of the mounting bracket 204 is aligned with the convex surface of the second end of the flexible coupling 208, the adjustment screw 250 is tightened. In an embodiment, in step 515 a top view is used (e.g. FIG. 6M) to visually confirm that the convex surface 259 of the second end 272 of the mounting bracket 204 is aligned with the convex surface 209 of the second end of the flexible coupling 208.

In an embodiment, in step 517 a backing 216b is removed from adhesive 217b on a second side of the fastener strip 210 (e.g. where a first side of the fastener strips 210 was secured to the mounting bracket 204 in step 509) and the protective shield 202 is secured to the adhesive 217b (e.g. above the opening 203). FIGS. 6N through 6P depict one embodiment of performing step 517. In an embodiment, FIG. 6P depicts that the adhesive 217b should be secured to the inside surface of the protective shield 202 and/or above the opening 203 and/or centered above the opening 203. In an embodiment, the adhesive 217b should be secured to the inside surface of the protective shield 202 at a position such that the convex surface 209 of the flexible coupling 208 engages and/or is aligned with the protective shield 202 adjacent the opening 203. In one embodiment, step 517 is performed so that the convex surface 209 of the flexible coupling 208 engages the inside surface of the protective shield 202, outside the opening 203. In an example embodiment, FIG. 60 depicts that a circle 290 and/or other marking (e.g. based on a shape of the perimeter of the second side of the flexible coupling 208) can be traced or drawn along the surface of the protective sheet 202, where the circle 290 encircles the opening 203 so that the user can use the circle 290 as a guide when aligning and engaging the inside surface of the protective shield 202 with the flexible coupling 208. Although the fastener strip 210 is used in the above embodiment of steps 509 and 517, in other embodiments any fastener appreciated by one of ordinary skill in the art can be employed to attach the mounting bracket 204 to the protective shield 202.

In an embodiment, in step 519 a length 264 of the mounting bracket 204 is adjusted to bring the protective shield 202 closer to the examiner 102 and create or enhance a seal between the protective shield 202 against the flexible coupling 208. FIGS. 6Q through 6S depict one embodiment of performing step 519. In an embodiment, the tool 212 is used to adjust the adjustment screw 250 and move the second end 272 of the mounting bracket 204 in a direction of the user 102. In an embodiment, as shown in FIG. 6R, this adjustment of the mounting bracket 204 towards the user 102 (e.g. the left direction viewing FIG. 6R) causes the seal between the flexible coupling 208 and the protective shield 202 to become air-tight since the adjustment of the mounting bracket 204 causes the protective shield 202 to also move toward the user 102 and thus enhances the seal between the protective shield 202 and the flexible coupling 208. The inventors of the present invention noticed that step 519 causes the seal between the flexible coupling 208 and the protective shield 202 to be airtight such that no viral or bacterial pathogens adjacent the opening 203 can pass between the protective shield 202 and the flexible coupling 208, further protecting the user 102.

FIG. 5B is a flow diagram that illustrates an example of a method 550 for installing the apparatus 400 of FIG. 4A to an optical assembly, according to an embodiment. In an embodiment, steps 551, 553 and 555 of the method 550 are similar to respective steps 501, 503 and 511 of the method 500. In another embodiment, steps 505, 507, 509 and steps 513 through 519 of the method 500 are excluded in the method 550, since they involve the mounting bracket 204 that is not used in the method 550.

In an embodiment, after performing steps 551, 553 and 555, the adaptive coupling 206 is secured to the housing of the optical assembly (e.g. housing of the ophthalmoscope 100) and the flexible coupling 208′ (e.g. with the magnets 402) is secured to the flange 240 of the adaptive coupling 206.

In an embodiment, unlike the method 500 that used the protective shield 202 without magnets, the protective shield 202 employed in the method 550 includes the magnets 404 arranged around the opening 203 of the protective shield 202.

In an embodiment, in step 557 the protective shield 202 is mounted to the housing of the optical assembly (e.g. housing of the ophthalmoscope 100) using the mounting fixture (e.g. adaptive coupling 205′). In an embodiment, in step 557 the protective shield 202 is attached to the adaptive coupling 205′ (e.g. to the flexible coupling 208′) such that the opening 203 is aligned with the optical axis 275 and the adaptive coupling 205′ forms an airtight seal around the opening 203 and/or between the housing of the optical assembly and the protective shield 202. In one embodiment, in step 557 the protective shield 202 is positioned relative to the adaptive coupling 205′ such that the magnets 404 on the protective shield 202 are aligned with the magnets 402 on the flexible coupling 208′. When the magnets 402, 404 are moved within a threshold distance, the magnets are attracted to each other which causes the protective shield 202 to contact and press against the flexible coupling 208′. In an example embodiment, the magnets 402, 404 attract each other such that upon the protective shield 202 pressing against the flexible coupling 208′, the opening 203 automatically aligns (e.g. centered) with the optical axis 275 of the optical assembly (FIG. 4F). In yet another example embodiment, the magnets 402, 404 attract each other such that when the protective shield 202 presses against the flexible coupling 208′, an airtight seal is formed by the adaptive coupling 205′ around the opening 203 and/or in the gap between the optical assembly and the protective shield 202. This advantageously maintains the sanitary barrier between the user and the patient, by extending the sanitary barrier to include the adaptive coupling 205′ on the rear side of the protective shield 202.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

What is claimed is:

1. An apparatus to provide a barrier for a user of an optical assembly including headgear and an optic within a housing, said apparatus comprising:
the barrier including a protective shield that defines an opening; and
a mounting fixture configured to attach the protective shield to the optical assembly such that the opening is aligned with an optical axis of the optic to provide an unobstructed path for light to the optic through the opening; and
an adaptive coupling positioned in a gap between the protective shield and the housing and configured to engage the protective shield adjacent the opening to form an airtight seal in the gap between the protective shield and the housing.

2. The apparatus as recited in claim 1, wherein the optic within the housing comprises one of the optic of a binocular indirect ophthalmoscope or the optic of a binocular indirect laser ophthalmoscope.

3. The apparatus as recited in claim 1, wherein the mounting fixture includes a centering feature that is configured to center the opening around the optical axis when the protective shield is attached to the optical assembly.

4. The apparatus as recited in claim 3, wherein the centering feature includes one or more first magnets arranged around the optical axis and one or more second magnets arranged around the opening such that upon engagement of the one or more first magnets with the one or more second magnets the opening is centered with the optical axis.

5. The apparatus as recited in claim 1, wherein the adaptive coupling includes a first side configured to secure to the housing and a second side opposite to the first side configured to engage the protective shield adjacent the opening such that the adaptive coupling forms the airtight seal between the housing and the protective shield.

6. The apparatus as recited in claim 5,
wherein the second side includes a plurality of first magnets arranged around the optical axis when the first side is secured to the housing; and
wherein the protective shield includes a plurality of second magnets that are arranged around the opening.

7. The apparatus as recited in claim 6, wherein the adaptive coupling is configured such that upon engagement of the plurality of first magnets to the plurality of second magnets the protective shield compresses against the second side of the adaptive coupling to form the seal between the housing and the protective shield.

8. The apparatus as recited in claim 6, wherein the plurality of first magnets are arranged around a perimeter of the second side.

9. The apparatus as recited in claim 8, wherein the plurality of first magnets are arranged around the perimeter of the second side to form a triangular arrangement and wherein the plurality of second magnets are arranged around the opening of the protective shield to form a triangular arrangement.

10. The apparatus as recited in claim 6, wherein the plurality of first magnets are embedded within a respective plurality of cavities defined by the second side and wherein the plurality of cavities are sized to receive the plurality of first magnets.

11. The apparatus as recited in claim 6, wherein the plurality of second magnets are attached around the opening on a front side of the protective shield opposite to a rear side of the protective shield facing the optical assembly when the protective shield is attached to the optical assembly.

12. The apparatus as recited in claim 5, wherein the adaptive coupling comprises:
a first portion with a first side that is configured to be secured to the housing and a second side opposite from the first side that comprises a flange; and
a second portion with a first side configured to engage the flange of the first portion and a second side opposite to the first side that is configured to engage the protective shield.

13. The apparatus as recited in claim 5, wherein the second side of the adaptive coupling and the protective shield each define an arcuate shape such that the second side with the arcuate shape is configured to engage the protective shield with the arcuate shape.

14. The apparatus as recited in claim 1, further comprising a headgear fixture to mount the housing to the headgear, wherein the mounting fixture is different from the headgear fixture and the mounting fixture is configured to attach the protective shield to the headgear independent of the headgear fixture.

15. The apparatus as recited in claim 14, wherein the headgear fixture mounts the housing to a hinge screw of the headgear and wherein the mounting fixture comprises a mounting bracket with a first end configured to be secured to the hinge screw and a second end opposite the first end configured to be secured to the protective shield.

16. The apparatus as recited in claim 15, wherein the second end of the mounting bracket includes a first fastener comprising one of a snap-together fastener, a temporary fastening strip and attachable fabric and the protective shield comprises a second fastener comprising one of the snap-together fastener, the temporary fastening strip and attachable fabric configured to secure to the first fastener.

17. The apparatus as recited in claim 15, wherein the housing is rotatable about the hinge screw and wherein the first end of the mounting bracket is configured such that the protective shield and the mounting bracket are independently rotatable about the hinge screw relative to the housing.

18. The apparatus as recited in claim 15, wherein the mounting bracket is configured with an adjustable length such that a separation between the headgear and the protective shield can be adjusted based on the adjustable length of the mounting bracket.

19. An apparatus to provide a barrier for a user of an optical assembly including headgear and an optic within a housing, said apparatus comprising:
the barrier including a protective shield that defines an opening; and
a mounting fixture separate from the protective shield and configured to attach the protective shield to the optical assembly such that the opening is aligned with an optical axis of the optic to provide an unobstructed path for light to the optic through the opening.

20. The apparatus as recited in claim 19, wherein the mounting fixture is an adaptive coupling positioned in a gap between the protective shield and the housing and configured to engage the protective shield adjacent the opening to form an airtight seal in the gap between the protective shield and the housing.

21. A method for installing the apparatus of claim 1 to the optical assembly, comprising the step of:
attaching, with the mounting fixture, the protective shield to the optical assembly such that the opening is aligned with the optical axis and the airtight seal is formed in the gap between the protective shield and the housing.

22. The method as recited in step 21, wherein the mounting fixture includes the adaptive coupling and wherein the attaching step comprises attaching the protective shield to the housing and wherein the attaching step further comprises forming, with the mounting fixture, the airtight seal between the housing and the protective shield such that the mounting fixture further defines the barrier.

23. The method as recited in claim 21, wherein the attaching step comprises centering, with a centering feature of the mounting fixture, the opening around the optical axis.

24. The method as recited in claim 23, wherein the centering step comprises engaging one or more first magnets of the centering feature arranged around the optical axis with one or more second magnets of the centering feature arranged around the opening.

25. The method as recited in claim 24, wherein the adaptive includes a plurality of the first magnets and wherein the method comprises:
securing a first side of the adaptive coupling to the housing such that the plurality of the first magnets at a second side of the adaptive coupling are arranged around the optical axis; and
securing a plurality of the second magnets arranged around the opening to the plurality of first magnets such that the protective shield compresses against the second side of the adaptive coupling and the adaptive coupling forms the airtight seal between the housing and the protective shield.

26. The method as recited in claim 25, wherein the securing the adaptive coupling to the housing includes:
securing a first side of an adapter plate, as a first portion of the adaptive coupling, to the housing, wherein a second side of the adapter plate includes a flange; and
securing a first side of a flexible coupling, as a second portion of the adaptive coupling, to the flange on the second side of the adapter plate, wherein a second side of the flexible coupling includes the plurality of the first magnets.

27. The method as recited in claim 24, further comprising aligning each of the one or more first magnets with a respective each of the one or more second magnets prior to the engaging step.

28. The method as recited in claim 21, further comprising:
mounting, with a headgear fixture, the housing to the headgear, wherein the mounting fixture is different from the headgear fixture;
and wherein the attaching step comprises attaching, with the mounting fixture, the protective shield to the headgear.

29. The method as recited in claim 28, wherein the attaching, with the mounting fixture, the protective shield to the headgear comprises:
attaching a first end of the mounting fixture to a hinge screw of the headgear; and
attaching a second end of the mounting fixture to the protective shield.

30. The method as recited in claim 29, wherein the headgear fixture mounts the housing to the hinge screw of the headgear and wherein the attaching the first end of the mounting fixture to the headgear comprises:
removing the hinge screw from a first opening in the headgear;
aligning a second opening in the first end of the mounting fixture with the first opening in the headgear; and
securing the hinge screw through the first opening and the second opening;
wherein upon performing the removing, aligning and securing steps, the protective shield and the mounting fixture are independently rotatable about the hinge screw relative to the housing.

31. The method as recited in claim 21, wherein the barrier further includes the adaptive coupling and wherein the method further comprises:
securing, a first side of the adaptive coupling, to the housing; and
securing, a second side of the adaptive coupling, to the protective shield adjacent the opening.

32. The method as recited in claim 31, wherein the adaptive coupling comprises, as a first portion, an adapter plate and, as a second portion, a flexible coupling and wherein the method further comprises:
securing, a first side of the adapter plate, to the housing;
securing, a first side of the flexible coupling, to a second side of the adapter plate; and
securing, a second side of the flexible coupling, to the protective shield adjacent the opening.

33. The method as recited in claim 32,
wherein the securing the first side of the adapter plate to the housing comprises removing a backing from an adhesive on the first side and applying the adhesive on the first side to the housing; and
wherein the securing the first side of the flexible coupling to the second side of the adapter plate comprises securing a flange along a perimeter of the second side of the adapter plate within an opening defined by the first side of the flexible coupling; and
wherein the securing the second side of the flexible coupling to the protective shield comprises positioning the protective shield, by adjusting a length of the second fixture, such that the protective shield compresses the flexible coupling and an airtight seal is formed between the protective shield and the second end of the flexible coupling around a perimeter of the flexible coupling.

34. The method as recited in claim 32, wherein the mounting fixture and the second side of the flexible coupling has an arcuate shape and wherein an inside surface of the protective shield has an arcuate shape, and wherein the method further comprises:
adjusting a length of the mounting fixture;
aligning the arcuate shape of the mounting fixture with the arcuate shape of the second side of the flexible coupling based on the adjusting step; and
securing the aligned arcuate shapes of the mounting fixture and the second side of the flexible coupling to the inside surface of the protective shield with the arcuate shape.

35. The method as recited in claim 32, further comprising placing a mark on the protective shield and wherein the securing the second side of the flexible coupling to the protective shield comprises aligning the second side of the flexible coupling with the mark on the protective shield.
